SEQUENCE LISTING (8 sequences)

SEQ ID NO: 1 - protein, 200 aa, Homo sapiens
MAFTEHSPLTPHRRDLCSRSIWLARKIRSDLTALTESYVKHQGLNKNINLDSADGMPVASTDQWSELTEAERLQENLQAYRTFHVLLARLLEDQQVHFTPTEGDFHQAIHTLLLQVAAFAYQIEELMILLEYKIPRNEADGMPINVGDGGLFEKKLWGLKVLQELSQWTVRSIHDLRFISSHQTGIPARGSHYIANNKKM

SEQ ID NO: 2 - protein, 200 aa, Artificial Sequence
  Synthetic
MAFTEHSPLTPHRRDLCSRSIWLARKIRSDLTALTESYVKHQGLNKNINLDSVDGVPVASTDRWSEMTEAERLQENLQAYRTFQGMLTKLLEDQRVHFTPTEGDFHQAIHTLLLQVSAFAYQLEELMVLLEQKIPENEADGMPATVGDGGLFEKKLWGLKVLQELSQWTVRSIHDLRVISSHQMGISALESHYGAKDKQM

SEQ ID NO: 3 - protein, 185 aa, Artificial Sequence
  synthetic
MAFTEHSPLTPHRRDLASRSIWLARKIRSDLTALTESYVKHQGLNKNINLDSADGMPVASTDRWSELTEAERLQENLQAYRTFHVLLARLLEDQQVHFTPTEGDFHQAIHTLLLQVAAFAYQIEELMILLEYKIPRNEADGMPINVGDGGLFEKKLWGLKVLQELSQWTVRSIHDLRFISSHQTG

SEQ ID NO: 4 - protein, 184 aa, Artificial Sequence
  Synthetic
AFTEHSPLTPHRRDLASRSIWLARKIRSDLTALTESYVKHQGLNKNINLDSADGMPVASTDRWSELTEAERLQENLQAYRTFHVLLARLLEDQQVHFTPTEGDFHQAIHTLLLQVAAFAYQIEELMILLEYKIPRNEADGMPINVGDGGLFEKKLWGLKVLQELSQWTVRSIHDLRFISSHQTG

SEQ ID NO: 5 - protein, 187 aa, Homo sapiens
MAFTEHSPLTPHRRDLASRSIWLARKIRSDLTALTESYVKHQGLNKNINLDSADGMPVASTDRWSELTEAERLQENLQAYRTFHVLLARLLEDQQVHFTPTEGDFHQAIHTLLLQVAAFAYQIEELMILLEYKIPRNEADGMPINVGDGGLFEKKLWGLKVLQELSQWTVRSIHDLRFISSHQTGIP

SEQ ID NO: 6 - protein, 187 aa, Homo sapiens
MAFTEHSPLTPHRRDLCSRSIWLARKIRSDLTALTESYVKHQGLNKNINLDSADGMPVASTDQWSELTEAERLQENLQAYRTFHVLLARLLEDQQVHFTPTEGDFHQAIHTLLLQVAAFAYQIEELMILLEYKIPRNEADGMPINVGDGGLFEKKLWGLKVLQELSQWTVRSIHDLRFISSHQTGIP

SEQ ID NO: 7 - protein, 187 aa, Homo sapiens
MAFTEHSPLTPHRRDLCSRSIWLARKIRSDLTALTESYVKHQGLNKNINLDSADGMPVASTDRWSELTEAERLQENLQAYRTFHVLLARLLEDQQVHFTPTEGDFHQAIHTLLLQVAAFAYQIEELMILLEYKIPRNEADGMPINVGDGGLFEKKLWGLKVLQELSQWTVRSIHDLRFISSHQTGIP

SEQ ID NO: 8 - protein, 187 aa, Homo sapiens
MAFTEHSPLTPHRRDLASRSIWLARKIRSDLTALTESYVKHQGLNKNINLDSADGMPVASTDQWSELTEAERLQENLQAYRTFHVLLARLLEDQQVHFTPTEGDFHQAIHTLLLQVAAFAYQIEELMILLEYKIPRNEADGMPINVGDGGLFEKKLWGLKVLQELSQWTVRSIHDLRFISSHQTGIP

(19) United States
(12) Patent Application Publication
(10) Pub. No.: US 2006/0058224 A1
(43) Pub. Date: Mar. 16, 2006

(54) Methods of treating obesity with combination therapeutics

(76) Inventors: George D. Yancopoulos, Yorktown Heights, NY (US); Stanley J. Wiegand, Croton-on-Hudson, NY (US); Mark W. Sleeman, Mahopac, NY (US); Ellen-Marie Koehler-Stec, Kent, NY (US)

Correspondence Address: REGENERON PHARMACEUTICALS, INC, 777 OLD SAW MILL RIVER ROAD, TARRYTOWN, NY, 10591, US

(21) Appl. No.: 11/229,426

(22) Filed: Sep. 15, 2005

Related U.S. Application Data
(60) Provisional application No. 60/609,926, filed on Sep. 15, 2004.

(51) Int. Cl. A61K 38/18; A61K 31/4439; A61K 31/426; A61K 31/365; A61K 31/155; A61K 31/16

(52) U.S. Cl. 514/2; 514/369; 514/625; 514/449; 514/340; 514/635

(57) ABSTRACT

Compositions and methods of treating obesity or obesity-related condition, including reducing body-weight, improving-diabetic parameters, metabolic syndrome. liver steatosis, and/or hypertension with a combination of CNTF or a CNTF-related molecule and a second agent which is a therapeutic molecule useful in the treatment of obesity, type II diabetes, or other obesity-related conditions.

### CROSS-REFERENCE TO RELATED APPLICATIONS

This application claims the benefit under 35 USC § 119(e) of U.S. Provisional 60/609,926 filed 15 Sep. 2004, which application is herein specifically incorporated by reference in its entirety.

### BACKGROUND OF THE INVENTION

1. Field of the Invention

The present invention relates to therapeutic combinations which include a CNTF-related polypeptide useful for the treatment of obesity and obesity related diseases or disorders.

2. Statement of Related Art

Ciliary neurotrophic factor (CNTF) is a protein that is required for the survival of embryonic chick ciliary ganglion neurons in vitro (Manthorpe et al., 1980, J. Neurochem. 34:69-75). Over the past decade, a number of biological effects have been ascribed to CNTF in addition to its ability to support the survival of ciliary ganglion neurons. CNTF has been cloned and synthesized in bacterial expression systems (Masiakowski et al. 1991 J. Neurosci. 57:1003-1012 and WO 91/04316, which are incorporated by reference in their entirety herein).

U.S. Pat. Nos. 6,472,178 and 6,565,869, both of which are herein specifically incorporated by reference in their entireties, describe modified ciliary neurotrophic factor (CNTF) molecules useful for treatment of a number of diseases, including neurological diseases, obesity, and diabetes. Human CNTF is shown in SEQ ID NO:1. Variants of human CNTF are shown as follows: hCNTF (Q63R) SEQ ID NO:2; Axokine™(“Ax-15”; hCNTF C17A Q63R Δ15) (SEQ ID NO:3 or SEQ ID NO:4); Ax-13 (hCNTF C17A Q63R Δ13) (SEQ ID NO:5); hCNTFΔ13 (SEQ ID NO:6); hCNTF Q63RΔ13 (SEQ ID NO:7); hCNTF C17AΔ13 (SEQ ID NO:8).

### SUMMARY OF THE INVENTION

The invention provides combinations of CNTF or a CNTF-related molecule and at least one agent useful in the treatment of obesity or an obesity-related condition, wherein the combination composition is useful for the treatment of obesity or an obesity-related condition.

In a first aspect, the invention features a pharmaceutical composition, comprising a CNTF molecule or a modified CNTF molecule in combination with at least one second agent, and a pharmaceutically acceptable carrier, wherein the second agent is a therapeutic molecule useful in the treatment of obesity.

In specific embodiments, the CNTF or CNTF-related molecule is human CNTF (SEQ ID NO:1), hCNTF (Q63R) (SEQ ID NO:2); Axokine™(“Ax-15”; hCNTF C17A Q63R Δ15) (SEQ ID NO:3 or SEQ ID NO:4); Ax-13 (hCNTF C17A Q63R Δ13) (SEQ ID NO:5); hCNTFΔ13 (SEQ ID NO:6); hCNTF Q63RΔ13 (SEQ ID NO:7); hCNTF C17AΔ13 (SEQ ID NO:8).

In specific embodiments, the second agent used in combination with a CNTF or CNTF-related molecule is one or more of sulfonylurea, biguanide metformin (e.g., Glucophage™, Bristol-Myers Squibb), and metformin variants, phentermine (Sigma), alpha-glucosidase inhibitors (e.g., Glucobay™, Precose™, Bayer), a thiazolidinedione such as troglitazone (Rezulin™, Warner-Lambert), rosiglitazone (Avandia™, SmithKline Beecham), pioglitazone (Actos™, Takeda/Lilly), repaglintide (NovoNorm™, Prandin™, Novo Nordisk), a small molecule such as MCC-555 (Mitsubishi), Targretin™ (Ligand Pharmaceuticals), bromocriptine (Ergoset™, Ergo Science), 5HT₂c receptor agonists (Cerebrus™, Roche), sibutramine, (Meridia™, Knoll), orlistat (Xenical™, Roche), leptin pathway therapeutics (metreleptin, Amgen), ghrelin antagonists, neuropeptide receptor antagonists, thermogenesis pathway therapeutics, PPARγ antagonists, aminoguanidine, AGE inhibitors, pimagedine, ALT-711 (Amgen), symlin (Pramlintide™, Exendin-4R, GLP-1, Amylin), HNF-4 modulators (Lingand Pharmaceuticals), MC4-R receptor modulators and other GPCRs (Millenium), small molecule MC4-R agonists, UCP modulators, and rimonabant (Acomplia™, Sanofi-Aventis) and other endocannabinoid receptor antagonists, bupropion (Wellbutrin™, Zyban™, Sanofi-Aventis), miglitol (Glyset™, Bayer), zonisamide (Zonegran™, Dainippon) and other calcium channel antagonists, topirqamate (Topamax™, Johnson & Johnson), bombesin, ATL 962 (Alizyme), AOD9604 (Metabolic), GW181771 and other CCK-A agonists (GSK), P57 (Phytopharm), PYY3-36 (Nastech Pharmaceuticals), AC 162352 (Amylin), SLV319 (Solvay), T71 (Tularik), ADP356 (Arena Pharmaceuticals), beta-3 AR agonists (L796568, Merck).

Preferred embodiments of the invention are those wherein the administration of the combination composition of the invention is subcutaneous, intramuscular, intradermal, intraperitoneal, intravenous, intranasal, epidural, and oral routes.

In a second aspect, the invention features a method of treating or reducing obesity, or an obesity-related condition, comprising administering a combination of CNTF or a CNTF-related molecule in combination with at least one second agent to a subject suffering from obesity or an obesity-related condition.

In specific embodiments, the obesity or obesity-related condition treated is a reduction in body weight, improvement in diabetic parameters such as fasting glucose and insulin levels, oral glucose tolerance, triglycerides and non-esterified free-fatty acids, type II diabetes, and liver steatosis, and hypertension. Generally, the combined therapeutics are useful for treatment of metabolic syndrome.

Other objects and advantages will become apparent from a review of the ensuing detailed description.

### BRIEF DESCRIPTION OF THE FIGURES

FIG. 1 is a graph of the change in body weight as a percentage of weight at Day 0 from Day 0 to Day 21 food intake (24 hours) over time (Days —1 to 21) for diet-induced obese (DIO) mice treated with 0.9% NaCl (4 μl/g, intraperitoneal (IP) injection) and placebo (4 μl/g subcutaneous (SC) injection; Group A: Control ═X); Axokine™ (0.03 mg/kg, SC; Group B=⋄); phentermine (10.0 mg/kg, IP; Group C=□); Axokine™ (0.03 mg/kg, SC) and phentermine (10.0 mg/kg, IP; Group D=□); and pair fed to group B=⋄).

FIG. 2 is a graph of 24 h food intake monitored for the duration of the study for the DIO mice treated as described in the legend of FIG. 1.

### DETAILED DESCRIPTION OF THE INVENTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only the appended claims.

As used in this specification and the appended claims, the singular forms “a”, “an”, and “the” include plural references unless the context clearly-dictates otherwise. Thus for example, a reference to “a method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference.

General Description

The invention is based in part on observation that administration of the modified CNTF molecule Axokine™ in combination with a second agent results in a far greater improvement in body weight and diabetic parameters such as fasting glucose and insulin levels, oral glucose tolerance, triglycerides and non-esterified free-fatty acids than can be achieved by comparable food restriction or with either agent alone.

Pharmaceutical Compositions

In one embodiment, the invention provides a pharmaceutical composition comprising CNTF or a modified CNTF molecule and, together or separately, a composition comprising one or more therapeutic agents, and a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical carriers are described in “Remington's Pharmaceutical Sciences” by E. W. Martin.

The composition of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the composition of the invention that will be effective for its intended therapeutic use can be determined by standard clinical techniques based on the present description. In addition, in vitro assays may optionally be employed to help identify optimal dosage ranges. Generally, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

For systemic administration, a therapeutically effective dose can be estimated initially from in vitro assays. For example, a dose can be formulated in animal models to achieve a circulating concentration range that includes the IC₅₀ as determined in cell culture. Such information can be used to more accurately determine useful doses in humans. Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the compounds that are sufficient to maintain therapeutic effect. In cases of local administration or selective uptake, the effective local concentration of the compounds may not be related to plasma concentration. One having skill in the art will be able to optimize therapeutically effective local dosages without undue experimentation.

The amount of compound administered will, of course, be dependent on the subject being treated, on the subject's weight, the severity of the affliction, the manner of administration, and the judgment of the prescribing physician. The therapy may be repeated intermittently while symptoms are detectable or even when they are not detectable. The therapy may also be provided in combination with other methods of treating obesity or obesity related conditions, for example, exercise, weight loss, reduction of alcohol intake, or improved control of a condition such as exercise, caloric restriction, etc.

Methods of Administration

The invention provides methods of treatment comprising administering to a subject an effective amount of a pharmaceutical composition(s) comprising CNTF or modified CNTF in in combination with one or more therapeutic agent(s) useful for treatment of obesity. The active components used in the method of the invention may be administered separately, simultaneously, sequentially, or together. Various delivery systems are known and can be used to administer the composition of the invention, e.g., encapsulation in liposomes, microparticles, microcapsules, recombinant cells capable of expressing the compound, receptor-mediated endocytosis (see, e.g., Wu and Wu, 1987, J. Biol. Chem. 262:4429-4432), construction of a nucleic acid as part of a retroviral or other vector, etc. Methods of introduction can be enteral or parenteral and include but are not limited to intradermal, intramuscular, intraperitoneal, intravenous, subcutaneous, intranasal, intraocular, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. Administration can be acute or chronic (e.g. daily, weekly, monthly, etc.) or in combination with other agents. Pulmonary administration can also be employed, e.g., by use of an inhaler or nebulizer, and formulation with an aerosolizing agent.

In another embodiment, the active agent can be delivered in a vesicle, in particular a liposome, in a controlled release system, or in a pump. In another embodiment where the active agent of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see, for example, U.S. Pat. No. 4,980,286), by direct injection, or by use of microparticle bombardment, or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression, by homologous recombination.

A composition useful in practicing the methods of the invention may be a liquid comprising an agent of the invention in solution, in suspension, or both. The term “solution/suspension” refers to a liquid composition where a first portion of the active agent is present in solution and a second portion of the active agent is present in particulate form, in suspension in a liquid matrix. A liquid composition also includes a gel. The liquid composition may be aqueous or in the form of an ointment.

An aqueous suspension or solution/suspension useful for practicing the methods of the invention may contain one or more polymers as suspending agents. Useful polymers include water-soluble polymers such as cellulosic polymers and water-insoluble polymers such as cross-linked carboxyl-containing polymers. An aqueous suspension or solution/suspension of the present invention is preferably viscous or muco-adhesive, or even more preferably, both viscous or mucoadhesive.

Other features of the invention will become apparent in the course of the following descriptions of exemplary embodiments which are given for illustration of the invention and are not intended to be limiting thereof.

### EXAMPLES

The following example is put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Effect of Axokine™ and Phentermine on Body Weight and Food Intake

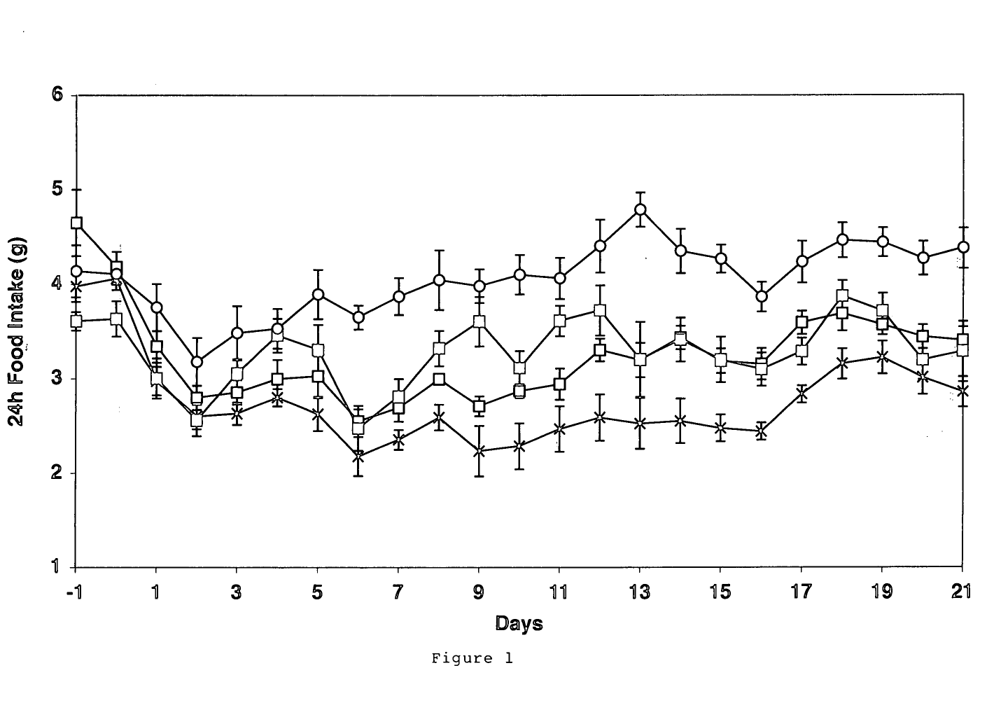

A study was conducted to determine the effect of the combination of phentermine and Axokine™ on body weight and food intake in a mouse model of obesity. 30 DIO (Diet-Induced Obesity; AKR/J mice fed a high fat diet) mice were divided into 5 equal groups (n=6 per group). Four groups were treated daily in the afternoon starting on Day 0 as follows: Group A (control): 0.9% NaCl (4 μl/g, intraperitoneal (IP) injection) and placebo (4 μl/g subcutaneous (SC) injection); Group B: Axokine™ (0.03 mg/kg, SC); Group C: phentermine (10.0 mg/kg, IP); Group D: Axokine™ (0.03 mg/kg, SC)+phentermine (10.0 mg/kg, IP). The sixth group, Group E was pair fed to Group B; each animal in this group received the average amount of food the Axokine™ treated animals consumed the previous day. This treatment paradigm continued until Day 21 (last injection on Day 20). As illustrated in FIG. 1, daily administration of Axokine™ or phentermine resulted in a significant decrease in body weight relative to control animals. After 21 days of treatment, animals receiving Axokine™ lost approximately 19% of their starting body weight; phentermine-treated mice lost about 15%, while control animals had a decrease of 2%. DIO mice receiving daily administration of Axokine™+phentermine lost approximately 26% of their starting body weight, an amount that was significantly greater than either Axokine™ alone or phentermine alone. Animals who were pair-fed to the Axokine™ group, lost weight at a rate similar to that of the Axokine™ group up to Day 11 of the study. After this time, their weight loss reached a plateau. Despite receiving less food than controls and the same amount as the Axokine™ group, they did not lose any additional weight.

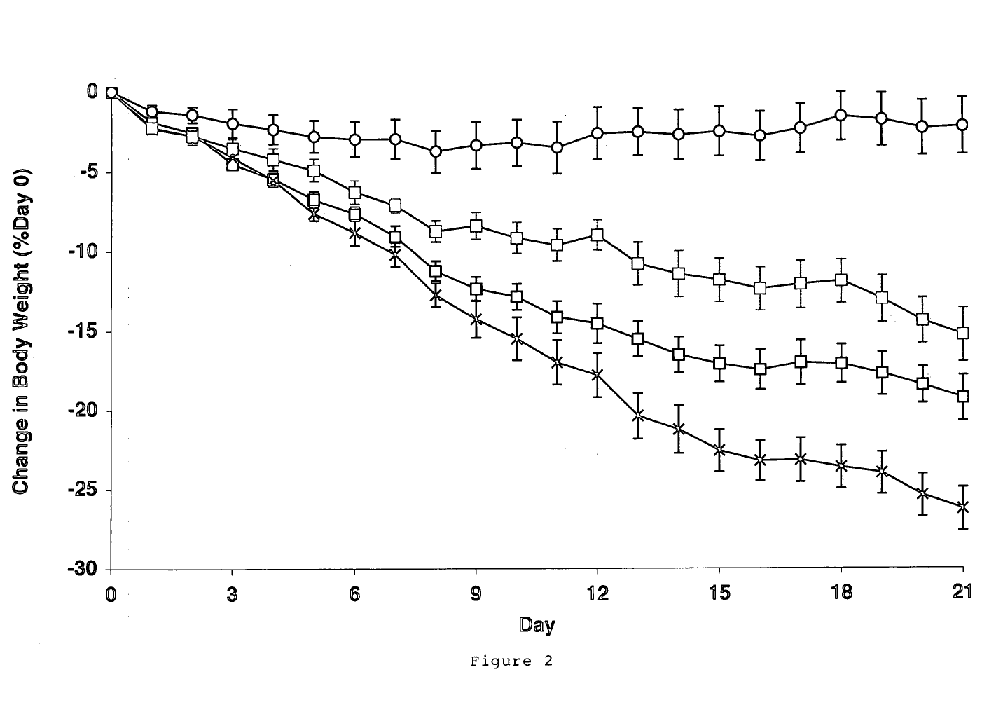

In terms of 24 h food intake (FIG. 2), all treatment groups had a decrease in food intake over the first 2 days of the study (Day 0 to Day 2) relative to their pre-study intake (Day —1). After the second day of the study, control animals resumed their typical eating pattern, consuming approximately 4 g of food a day. Animals who received Axokine™ or were pair-fed, maintained this decreased food intake of about 2.75 g per day until Day 12 when they began to consume more food (3.5 g at the end of the study).

Phentermine treated mice also had decreased food intake relative to control but the overall pattern was more variable. As expected, the combination of Axokine™ and phentermine also resulted in a decreased food consumption that was noticeably different than either compound alone, starting around Day 9 of the study. Taken together, the results of the study demonstrate that the combination of Axokine™ and phentermine can cause a greater degree of weight loss than either agent by itself and this is due in part to a reduction in food intake.

### Example 2

### Effect of Axokine™ and Rimonabant on Food Intake and Weight Loss

Rimonabant (SR-141716 or N-piperidine-5-(4-chlorophenyl)-1-(2,4-dichloropheyl)-4-methylpyrazole-3-carbozamide) is a CB1 receptor antagonist. A study is conducted of the effect of Axokine™ alone, rimonabant alone, or Axokine™+rimonabant on food intake and weight loss on 25 DIO (Akr/J) mice as described in Example 1. The mice are divided into 5 equal groups treated as follows: Group A (n=5) control (200 μl PBS subcutaneous); Group B (n=5): 0.03 mg/kg Axokine™ (subcutaneous injection); Group C (n=5) 1.0-10.0 mg/kg rimonabant (intraperitoneal injection); Group D (n=5) 0.03 mg/kg Axokine™ (subcutaneous injection)+1.0 mg/kg rimonabant (intraperitoneal injection); Group E (n=5) pair fed to Group B+200 μl PBS (subcutaneous injection). Food intake and body weight are measured daily over the 21 day treatment period. Terminal serum chemistry analyis (22 point analysis) is conducted at the end of the treatment period.

We claim:

1. A pharmaceutical composition comprising a first CNTF or CNTF-related molecule in combination with at least one second agent, and a pharmaceutically acceptable carrier, wherein the second agent is a therapeutic molecule useful in the treatment of obesity or an obesity-related condition.

2. The pharmaceutical composition of claim 1, wherein the CNTF or CNTF-related molecule is selected from the group consisting of human CNTF (SEQ ID NO:1), hCNTF (Q63R) (SEQ ID NO:2); Axokine™(“Ax-15”; hCNTF C17A Q63R Δ15) (SEQ ID NO:3 or SEQ ID NO:4); Ax-13 (hCNTF C17A Q63R Δ13) (SEQ ID NO:5); hCNTFΔ13 (SEQ ID NO:6); hCNTF Q63RΔ13 (SEQ ID NO:7); and hCNTF C17AΔ13 (SEQ ID NO:8).

3. The pharmaceutical composition of claim 1, wherein the second agent is selected from the group consisting of sulfonylurea, biguanide mefformin and mefformin variants, alpha-glucosidase inhibitors, a thiazolidinedione, rosiglitazone, pioglitazone, repaglintide, a small molecule, bromocriptine, an 5HT_{2c} receptor agonist, sibutramine, orlistat, a leptin pathway therapeutic, a ghrelin antagonist, a neuropeptide receptor antagonist, a thermogenesis pathway therapeutic, a PPARγ antagonist, aminoguanidine, an AGE inhibitor, pimagedine, ALT-711, symlin, a HNF-4 modulator, a MC4-R receptor modulator, a GPCR, small molecule MC4-R agonist, a UCP modulator, rimonabant, an endocannabinoid receptor antagonist, bupropion, miglitol, zonisamide, a calcium channel antagonist, topirqamate, bombesin, ATL 962, AOD9604, GW181771, a CCK-A agonist, P57, PYY3-36, AC 162352, SLV319, T71, ADP356, and a beta-3 AR agonist.

4. The pharmaceutical composition of claim 1, wherein administration is subcutaneous, intramuscular, intradermal, intraperitoneal, intravenous, intranasal, epidural, and/or oral.

5. A pharmaceutical composition comprising a first CNTF or CNTF-related molecule in combination with at least one second agent, and a pharmaceutically acceptable carrier,
wherein the CNTF or CNTF-related molecule is selected from the group consisting of human CNTF (SEQ ID NO:1), hCNTF (Q63R) (SEQ ID NO:2); Axokine™(“Ax-15”; hCNTF C17A Q63R Δ15) (SEQ ID NO:3 or SEQ ID NO:4); Ax-13 (hCNTF C17A Q63R Δ13) (SEQ ID NO:5); hCNTFΔ13 (SEQ ID NO:6); hCNTF Q63RΔ13 (SEQ ID NO:7); and hCNTF C17AΔ13 (SEQ ID NO:8), and
wherein the second agent is selected from the group consisting of sulfonylurea, biguanide metformin and metformin variants, alpha-glucosidase inhibitors, a thiazolidinedione, rosiglitazone, pioglitazone, repaglintide, a small molecule, bromocriptine, an 5HT_{2c} receptor agonist, sibutramine, orlistat, a leptin pathway therapeutic, a ghrelin antagonist, a neuropeptide receptor antagonist, a thermogenesis pathway therapeutic, a PPARγ antagonist, aminoguanidine, an AGE inhibitor, pimagedine, ALT-711, symlin, a HNF-4 modulator, a MC4-R receptor modulator, a GPCR, small molecule MC4-R agonist, a UCP modulator, rimonabant, an endocannabinoid receptor antagonist, bupropion, miglitol, zonisamide, a calcium channel antagonist, topirqamate, bombesin, ATL 962, AOD9604, GW181771, a CCK-A agonist, P57, PYY3-36, AC 162352, SLV319, T71, ADP356, and a beta-3 AR agonist.

6. A method of treating or reducing obesity, or an obesity-related condition, comprising administering a combination of CNTF or a CNTF-related molecule in combination with at least one second agent to a subject suffering from obesity or an obesity-related condition.

7. The method of claim 6, wherein the obesity or obesity-related condition treated is a reduction in body weight, improvement in diabetic parameters, metabolic syndrome, liver steatosis, and hypertension.

8. The method of claim 7, wherein the diabetic parameter is one or more of fasting glucose and insulin levels, oral glucose tolerance, triglycerides and non-esterified free-fatty acids, and type II diabetes.

9. The method of claim 6, wherein the CNTF or CNTF-related molecule is selected from the group consisting of human CNTF (SEQ ID NO:1), hCNTF (Q63R) (SEQ ID NO:2); Axokine™(“Ax-15”; hCNTF C17A Q63R Δ15) (SEQ ID NO:3 or SEQ ID NO:4); Ax-13 (hCNTF C17A Q63R Δ13) (SEQ ID NO:5); hCNTFΔ13 (SEQ ID NO:6); hCNTF Q63RΔ13 (SEQ ID NO:7); and hCNTF C17AΔ13 (SEQ ID NO:8).

10. The method of claim 6, wherein the second agent is selected from the group consisting of sulfonylurea, biguanide, metformin and metformin variants, alpha-glucosidase inhibitors, a thiazolidinedione, rosiglitazone, pioglitazone, repaglintide, a small molecule, bromocriptine, an 5HT_{2c} receptor agonist, sibutramine, orlistat, a leptin pathway therapeutic, a ghrelin antagonist, a neuropeptide receptor antagonist, a thermogenesis pathway therapeutic, a PPARγ antagonist, aminoguanidine, an AGE inhibitor, pimagedine, ALT-711, symlin, a HNF-4 modulator, a MC4-R receptor modulator, a GPCR, small molecule MC4-R agonist, a UCP modulator, rimonabant, an endocannabinoid receptor antagonist, bupropion, miglitol, zonisamide, a calcium channel antagonist, topirqamate, bombesin, ATL 962, AOD9604, GW181771, a CCK-A agonist, P57, PYY3-36, AC 162352, SLV319, T71, ADP356, and a beta-3 AR agonist.
